(19) United States
(12) Patent Application Publication
(10) Pub. No.: US 2012/0251593 A1
(43) Pub. Date: Oct. 4, 2012

(54) SILK FIBROIN-DECORIN SCAFFOLDS

(75) Inventors: Anshu B. Mathur, Missouri City, TX (US); Charles E. Butler, Houston, TX (US); Vishal Gupta, Pearland, TX (US); Nadja K. Burns, Houston, TX (US)

(73) Assignee: THE BAOARD OF REGENTS OF THE UNIVERSITY OF TEXAS SYSTEM, Austin, TX (US)

(21) Appl. No.: 13/498,640

(22) PCT Filed: Sep. 28, 2010
(86) PCT No.: PCT/US2010/050541

§ 371 Date: Jun. 11, 2012

Related U.S. Application Data
(60) Provisional application No. 61/246,448, filed on Sep. 28, 2009.

(51) Int. Cl. A61K 47/42; A61P 1/00; A61K 9/00

(52) U.S. Cl. 424/400; 514/773

(57) ABSTRACT

Disclosed are silk fibroin scaffolds that are fabricated with decorin proteoglycan, and methods of using these scaffolds in the repair of tissue defects in subjects. The scaffolds have biomechanical properties which makes them suitable for patient-specific design for defects where strong tensile strength is required, such as musculofascia reconstruction.

The present application claims benefit of priority to U.S. Provisional Application Ser. No. 61/246,448 filed Sep. 28, 2009, the entire contents of which are hereby incorporated by reference.

The invention was made with government support under Grant Nos. R21AG026477 and R01AG034658 awarded by the National Institutes of Health. The government has certain rights in the invention.

### BACKGROUND OF THE INVENTION

1. Field of the Invention

The present invention relates generally to the fields of tissue scaffolds (bioprosthetics) and the repair of tissue defects in a subject. More particularly, it concerns silk fibroin-decorin scaffolds and application of these scaffolds in the repair of tissue defects.

2. Description of Related Art

Approximately 200,000 ventral (incisional) abdominal wall hernias are repaired annually in the United States. When prosthetic mesh is used for repair, the incidence of recurrence is reduced from 33% to 0-10% (Gobin et al., 2006). Some currently commercially available materials for ventral hernia repair include synthetic materials such as polypropylene mesh (Prolene, Ethicon, Sommerville, N.J.) and bioprosthetic materials such as human acellular dermal matrix (AlloDerm®, LifeCell Corp., Branchburg, N.J.). Although polypropylene mesh has a strong mechanical strength that helps it withstand intra-abdominal pressures, it also forms a surrounding scar with adhesions leading to bowel obstruction, perforation, enterocutatneous fistulae, and pain (Butler, 2006; Butler et al., 2001; Butler and Prieto, 2004). Biological materials tend to cause less and weaker adhesions, however, they are more expensive and are often available only in limited sizes (Gobin et al., 2006; Butler et al., 2005). Hence a variety of materials are available commercially, but have serious drawbacks, which may cause patient morbidity. Thus, there is a need of tissue engineered material, which has no scarring and good integration with the abdominal tissue for reconstructive surgery.

In a previous study, 75:25 silk fibroin (SF) and chitosan (CS) blend (SFCS, 75% SF and 25% CS) scaffolds was applied in the repair of abdominal wall musculofascia in an in vivo guinea pig model (Gobin et al., 2006). SF has attractive features for biomedical engineering such as permeability to oxygen and water, cell adhesion and growth characteristic, low thrombogenicity, low inflammatory response, protease susceptibility, and high tensile strength with flexibility. The other component CS promotes wound healing (Gobin et al., 2006). The mean ultimate tensile strength (UTS) of the guinea pig native abdominal wall was found to be 130 kPa (Gobin et al., 2006). One limitation of the 75:25 SFCS scaffold in the previous study was that the pre-implant UTS was only 24 kPa, suggesting that these scaffolds might not be suitable for abdominal wall repair in humans (Gobin et al., 2005). Four weeks after repair, the UTS of regenerated abdominal wall was 628 kPa (Gobin et al., 2006).

SF has the properties similar to the extracellular matrix (ECM) protein collagen, which is the most abundant protein in human body. Another ECM component decorin interact with collagen and enhance the tensile strength in tissues such as tendon. Decorin is a small leucine-rich proteoglycan with a core protein of ˜40 kDa. Decorin molecule is made of three domains: an N-terminal region possesses a single chondroitin/dermatan sulfate side chain and a distinct pattern of Cys residues; a central region is composed of ten leucine-rich repeats which are believed to interact with other proteins, including collagen and transforming growth factor-β (TGF-β); and another Cys-rich C-terminal region (Iozzo, 1998; Reed and Iozzo, 2003). Decorin affect collagen fibrillogenesis, growth factor modulation, and regulation of cellular growth (Reed and Iozzo, 2003; Ferdous and Grande-Allen, 2006; Liao and Vesely, 2007).

Thus, there is the need for scaffolds that have an improved pre-implant tensile strength that approaches that of the native abdominal wall.

### SUMMARY OF THE INVENTION

The present invention in part provides for silk fibroin (SF) scaffolds that are fabricated with decorin proteoglycan. Fabrication of SF scaffolds with decorin proteoglycan allows for significantly improved bioengineering properties compared to SFCS blend scaffolds. These improved properties include increased pre-implant tensile strength that provides for repair of tissue defects in humans. The entangled fibrillar structure of the SF-decorin contributes to the increased mechanical strength of the SF scaffold, making the scaffolds suitable for repair of defects where high tensile strength is needed, including musculofascia defects.

Some embodiments of the present invention generally concern biocompatible scaffolds that include a silk fibroin polypeptide and a decorin proteoglycan in contact with the silk fibroin polypeptide. The scaffolds are suitable for implantation in a subject for tissue regeneration.

The ratio of decorin proteoglycan:silk fibroin polypeptide may be any ratio. In some embodiments, the ratio of decorin proteoglycan:silk fibroin polypeptide in the scaffold ranges from about 1:100 (w/w) to about 1:1×10⁸ (w/w). In further embodiments, the ratio of decorin proteoglycan:silk fibroin polypeptide in the scaffold ranges from about 1:100 (w/w) to about 1:1×10⁶ (w/w). In still further embodiments, the ratio of decorin proteoglycan:silk fibroin polypeptide in the scaffold ranges from about 1:100 (w/w) to about 1:1×10⁴ (w/w). In even further embodiments, the ratio of decorin proteoglycan:silk fibroin polypeptide in the scaffold ranges from about 1:100 (w/w) to about 1:1000 (w/w).

The silk fibroin may be genetically engineered, chemically synthesized, or obtained from natural sources. In particular embodiments the silk fibroin is from the silkworm Bombyx mori (hereinafter “silk fibroin” abbreviated as SF; SEQ ID NO:1; GenBank Accession No. AAL83649). Other examples of fibroins associated with silk from other insects such as spider are contemplated for inclusion in the scaffolds of the present invention. Other examples of fibroins include fibroin from Antipaluria urichi (GenBank Accession No. ACJ04053; SEQ ID NO:2); fibroin from Oecophylla smaragdina (GenBank Accession No. ABW21705; SEQ ID NO:3); fibroin from Oecophylla smaragdina (GenBank Accession No. ABW21703; SEQ ID NO:4); fibroin from Mymecia forficata (GenBank Accession No. ABW21701; SEQ ID NO:5); and fibroin from Bombus terrestris (GenBank Accession No. ABW21697; SEQ ID NO:6). The fibroin may be produced from genetically engineered cells in vivo.

In some embodiments, the SF polypeptide comprises a consecutive series of at least 10, 20, 30, 50, 75, 100, 125, 150, 200, 225, 250, or the full-length amino acid sequence of silk fibroin (262), or any range of numbers of consecutive sequences of amino acids derivable herein. Thus, for example, a SF polypeptide may comprise between 10 and 262, between 20 and 250, between 30 and 220, between 40 and 200, between 50 and 180, or between 60 and 120 consecutive amino acids of SEQ ID NO:1. The fibroin polypeptide may include one or more additional amino acid residues at the C-terminus or N-terminus of the consecutive sequence of amino acids of SEQ ID NO:1. In some embodiments, the fibroin polypeptide has at least 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, 99% or greater sequence homology to a known fibroin protein, such as SEQ ID NO:1.

The decorin proteoglycan can comprise any number of consecutive amino acids of a full-length decorin amino acid sequence as discussed in the specification below. Isoforms of the full-length amino acid sequence of human decorin include SEQ ID NO:7 (GenBank Accession No. AAA52301), SEQ ID NO:8 (GenBank Accession No. AAB60901), SEQ ID NO:9 (GenBank Accession Number AAH05322), SEQ ID NO:10 (GenBank Accession No. AAV38603), and SEQ ID NO:11 (GenBank Accession No. AAL92176).

The scaffolds set forth herein may include one or more therapeutic agents. A therapeutic agent may be in contact with the surface of the scaffold, such as coated on the surface, or it may be incorporated into the scaffold matrix. Non-limiting examples are an antimicrobial agent, an anti-inflammatory agent, an immunosuppressant, or a growth factor.

The scaffold may be in a variety of shapes and sizes for the particular indication. In addition, the tissue scaffolds can be produced in three-dimensional forms to facilitate sizing. In particular embodiments the scaffold is configured as a sheet. The sheet may be of any thickness. For example, the thickness may be between about 0.1 mm to about 1 cm. In further embodiments, the thickness is between about 0.1 mm to about 5 mm.

Other aspects of the present invention concern methods of making any of the aforementioned biocompatible scaffolds. In some embodiments, the method includes (a) preparing a composition comprising a silk fibroin polypeptide, a decorin proteoglycan, and a solvent to create a blend; (b) placing the blend onto a surface; and (c) drying the blend to remove some or all of the solvent, wherein a biocompatible scaffold is formed. In some embodiments, the method further includes the step of removing the scaffold of (c) from the surface. Some embodiments further include (d) contacting the scaffold of (c) with a composition comprising an alcohol. The alcohol may be any alcohol known to those of ordinary skill in the art. Non-limiting examples include methanol and ethanol. In some embodiments, following contacting the scaffold with a composition comprising an alcohol, the scaffold is contacted with a solution of phosphate buffered saline. The scaffold can be dried and stored for later use. It may be stored in contact with a solution, such as phosphate buffered saline.

Other embodiments of the invention generally concern methods of treating a tissue defect in a subject that involve contacting the subject with one of the biocompatible scaffolds of the present invention. The subject can be any subject, such as a mammalian subject. Non-limiting examples of mammalian subjects include a human, a primate, a cow, a horse, a sheep, a goat, a dog, a cat, a rabbit, a dog, or a rodent. In particular embodiments the subject is a human. The human, for example, may be a subject with a tissue defect. The defect may be a defect in abdominal wall musculofascia such as a hernia.

The scaffolds can be used for soft tissue reinforcement or repair of a tissue defect involving any part of a subject. The tissue defect may be a defect in abdominal wall musculofascia such as a hernia, a surgical defect in tissue, a traumatic defect, a congenital defect or other defect. These uses and applications of the present scaffolds are illustrative of several potential uses and should not be construed as limiting the types of uses and applications for the scaffolds prepared by the methods and processes described herein. In certain embodiments, the scaffold is implanted in a subject as part of a surgical procedure directed at repairing a musculofascia defect in a subject. Non-limiting examples of musculofascia defects include an abdominal hernia, an inguinal hernia, a hiatal hernia, a diaphragmatic hernia, an anal hernia, a femoral hernia, an umbilical hernia, and an incisional hernia.

Other embodiments of the present invention concern kits comprising a scaffold of the present invention in a sealed container.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. Representative stress versus strain curves for SF and SFCS scaffolds as well as SF- and SFCS-decorin scaffolds at 7.5 and 28.6 μg/mL decorin concentrations.

FIG. 2A, 2B, 2C. Mechanical properties comparison across SF- and SFCS-decorin concentrations. (a) Elastic modulus; *p<0.05, **p<0.01 and ***p<0.001 vs. SFCS control, ^{#}p<0.05 vs. SFCS-28.6 μg/mL decorin, ^{!}p<0.01 vs. SF-1.9 μg/mL decorin, ^{@}p<0.01 vs. SF-3.8 μg/mL decorin, ^{$}p<0.05 vs. SF-5.6 μg/mL decorin, ^{%}p<0.05 vs. SF-7.4 μg/mL decorin, ̂p<01 vs. SF-16.6 μg/mL decorin, ^{&}p<0.001 vs. SF-28.6 μg/mL decorin, (b) Ultimate tensile strength; *p<0.05 and **p<0.01 vs. SF-28.6 μg/mL decorin, ^{!}p<0.05, ^{!!}p<0.01 and ^{!!!}p<0.001 vs. SFCS control, ^{#}p<0.01 vs. SFCS-1.9 μg/mL decorin, ^{@}p<0.01 vs. SFCS-3.8 μg/mL decorin, ^{$}p<0.05 vs. SFCS-7.4 μg/mL decorin, ^{%}p<0.05 vs. SFCS-16.6 μg/mL decorin, ̂p<01 vs. SFCS-28.6 μg/mL decorin, (c) Strain at failure; *p<0.05 vs. SF-16.6 μg/mL decorin, ^{#}p<0.05 ^{##}p<0.01 vs. SF-28.6 μg/mL decorin, ^{@}p<0.01 vs. SFCS-3.8 μg/mL decorin, ^{$}p<0.01 vs. SFCS control, ^{%}p<0.05 vs. SFCS-7.4 μg/mL decorin.

FIG. 3. Representative Raman spectra for SF and varying decorin concentrations in SF.

### DESCRIPTION OF ILLUSTRATIVE EMBODIMENTS

The present invention is based on the finding that silk fibroin (SF) scaffolds that are fabricated with decorin proteoglycan have improved mechanical strength for application in humans. The improvement in mechanical strength of SF scaffolds with decorin proteoglycan provides a viable solution in developing a patient-specific design for musculofascia reconstruction.

### A. SILK FIBROIN GENERALLY

Silk, as the term is generally known in the art, means a filamentous fiber product secreted by an organism such as a silkworm or spider. Silks produced from insects, namely (i) Bombyx mori silkworms, and (ii) the glands of spiders, typically Nephilia clavipes, are the most often studied forms of the material; however, hundreds to thousands of natural variants of silk exist in nature. Fibroin is produced and secreted by a silkworm's two silk glands.

Silkworm silk has been used in biomedical applications for over 1,000 years. The Bombyx mori species of silkworm produces a silk fiber (known as a “bave”) and uses the fiber to build its cocoon. The bave, as produced, includes two fibroin filaments or “broins,” which are surrounded with a coating of gum, known as sericin—the silk fibroin filament possesses significant mechanical integrity. When silk fibers are harvested for producing yarns or textiles, the sericin is partially dissolved and then resolidified to create a larger silk fiber structure having more than two broins mutually embedded in a sericin coating.

As used herein, the term “silk fibroin” pertains to silkworm fibroin. SF may be obtained from any source known to those of ordinary skill in the art. For example, SF may be obtained from a solution containing a dissolved silkworm silk from Bombyx mori. In the alternative, the SF suitable for use in the present invention can be obtained from a solution containing a genetically engineered silk.

The SF can be prepared by any conventional method known to one skilled in the art. For example, B. mori cocoons may be boiled in an aqueous solution. The cocoons are rinsed, for example, with water to extract the sericin proteins and the extracted silk is dissolved in an aqueous salt solution. The salt is consequently removed using, for example, dialysis. The SF may be produced using organic solvents. Such methods have been described, for example, in Li et al. (2001); Nazarov et al. (2004). SF may also be obtained from any of a number of commercial sources known to those of ordinary skill in the art.

Additional information concerning the production of silk fibroin can be found in U.S. Patent App. Pub. No. 20080176960, 20070187862, 20060019348, 20050260706, 20030165548, herein specifically incorporated by reference.

### B. DECORIN

Decorin is a member of the leucine-rich repeat (LRR) protein family and is composed of a 36.5 kDa core protein substituted with a glycosaminoglycan chain on a N-terminal Ser-Gly site (Krusius and Ruoslahti, 1986). The core protein contains leucine rich repeats flanked by disulfide bond-stabilized loops on both sides. It contains additional sites for glycosylation (N-linked glycosylation sites) within the leucine-rich repeats. The glycosaminoglycan chain backbone is composed of repeated disaccharide units of N-acetylgalactosamine and glucuronic acid. The molecular mass of decorin is about 75 KDa.

### C. POLYPEPTIDES

In certain embodiments, the present invention concerns scaffolds that include silk fibroin polypeptides and decorin polypeptides. As used herein, the term “polypeptide” refers to a consecutive series of two or more amino acids.

In certain embodiments the size of at least SF polypeptide or decorin polypeptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 260, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues, or any range of amino acid residues derivable therein (e.g., about 200 to about 2500 amino acid residues).

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moiety. In particular embodiments, the sequence of residues of the protein or peptide may be interrupted by one or more non-amino acid moieties.

Accordingly, the term “polypeptide” encompasses amino acid sequences comprising at least one of the 20 common amino acids found in naturally occurring proteins, or at least one modified or unusual amino acid, including but not limited to Aad, 2-Aminoadipic acid; EtAsn, N-Ethylasparagine; Baad, 3-Aminoadipic acid, Hyl, Hydroxylysine; Bala, β-alanine, β-Amino-propionic acid; AHy1, allo-Hydroxylysine; Abu, 2-Aminobutyric acid; 3Hyp, 3-Hydroxyproline; 4Abu, 4-Aminobutyric acid, piperidinic acid; 4Hyp, 4-Hydroxyproline; Acp, 6-Aminocaproic acid, Ide, Isodesmosine; Ahe, 2-Aminoheptanoic acid; AIle, allo-Isoleucine; Aib, 2-Aminoisobutyric acid; MeGly, N-Methylglycine, sarcosine; Baib, 3-Aminoisobutyric acid; MeIle, N-Methylisoleucine; Apm, 2-Aminopimelic acid; MeLys, 6-N-Methyllysine; Dbu, 2,4-Diaminobutyric acid; MeVal, N-Methylvaline; Des, Desmosine; Nva, Norvaline; Dpm, 2,2′-Diaminopimelic acid; Nle, Norleucine; Dpr, 2,3-Diaminopropionic acid; Urn, Ornithine; and EtGly, N-Ethylglycine.

Proteins or peptides may be made by any technique known to those of skill in the art, including the expression of polypeptides through standard molecular biological techniques, the isolation of polypeptides from natural sources, or the chemical synthesis of polypeptides. Alternatively, various commercial preparations of SF polypeptides are known to those of skill in the art.

### D. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Effect of Decorin on Silk Fibroin Based Scaffold Structure and Mechanical Properties

### Materials and Methods

Scaffold Preparation.

The preparation of pure SF from silk (donated from Dr. S. Hudson, TECS, N. Carolina State University, Raleigh, N.C.) and 75:25 SFCS blend has been described in detail by Gobin et. al., 2005. A 100 μg/mL solution of decorin (Sigma-Aldrich, St. Louis, Mo.) was prepared by adding 5 mL phosphate buffer solution (PBS, 1× without calcium and magnesium) to 0.5 mg decorin. Varying amounts of this stock solution were added to SF or SFCS solution to make final volume of 5 mL in concentrations ranging from 1.9 to 56.0 μg/mL decorin. Controls of 5 mL of SF or SFCS were also prepared. The solutions were then poured into plastic petri dishes (35 mm diameter). These petri dishes were set into larger dishes containing 99.9% ethanol and frozen overnight at −80° C. freezer followed by lyophilization for 2-3 days. The dry SFCS-decorin samples were crystallized with 50:50 (v/v) methanol:sodium hydroxide (1N) and the dry SF-decorin samples, with a 50% methanol solution for 15 min. The SFCS-decorin samples had the methanol:sodium hydroxide solution replaced with 1N NaOH overnight and the SF-decorin samples had the methanol solution replaced with PBS overnight. The samples were incubated in PBS with solution changes every 4 hours until the pH had equilibrated to 7 (Gobin et al., 2005). Five scaffolds were prepared for each condition.

Uniaxial Tensile Macroscopic Mechanical Testing.

Elastic modulus, UTS, and strain at failure (ε_{failure}) were calculated from stress-strain data measured using an EnduraTec ELF 3200 (Bose, Minnetonka, Minn.). Scaffold samples were tested with a 50-g load cell (Honeywell Sensotec, Columbus, Ohio) at 500 μm/sec strain rate.

Scanning Electron Microscopy (SEM).

Dehydrated samples were mounted on to double-stick carbon tabs (Ted Pella. Inc., Redding, Calif.), which have been previously mounted onto aluminum specimen mounts (Electron Microscopy Sciences, Ft. Washington, Pa.). The samples were then coated under vacuum using a Balzer MED 010 evaporator (Technotrade International, Manchester, N.H.) with platinum alloy for a thickness of 25 nm. The samples were transferred to a desiccator for examination at a later date. Samples were examined in a JSM-5910 scanning electron microscope (JEOL, USA, Inc., Peabody, Mass.) at an accelerating voltage of 5 kV.

Raman Spectroscopy.

Scaffolds after PBS wash were assessed at room temperature for Raman spectroscopy. Raman Systems R-3000 QE (Raman Systems Inc.™, Austin, Tex.) with a 785 nm class Mb laser and RSI-Scan version 1.3.83 software were used for Raman spectroscopic analysis. The measurement time of a single spectrum was typically around 20 seconds. No sample deterioration was noted under these conditions.

Statistical Analysis.

Data sets were compared using two-tailed, unpaired t tests in GraphPad Instat 3 program and p values of less than 0.05 were considered significant. All the data was presented as mean±standard error of mean.

### Results

SFCS-Decorin and SF-Decorin Scaffold Mechanical Properties.

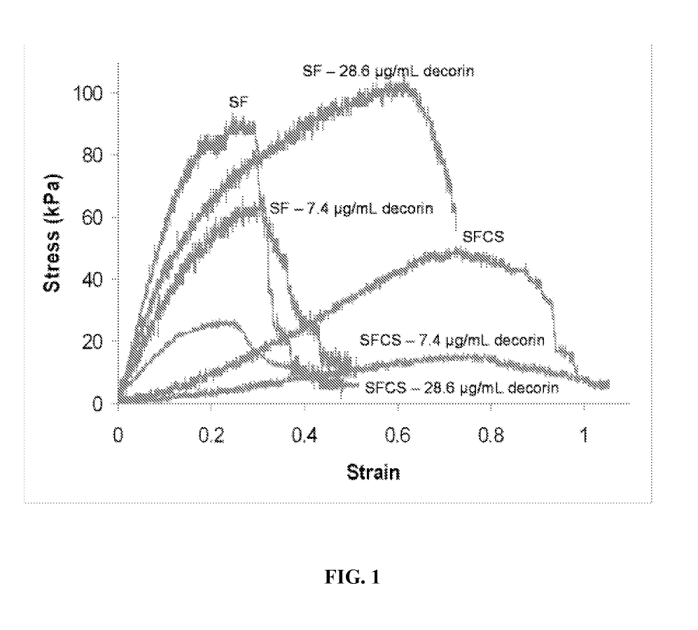

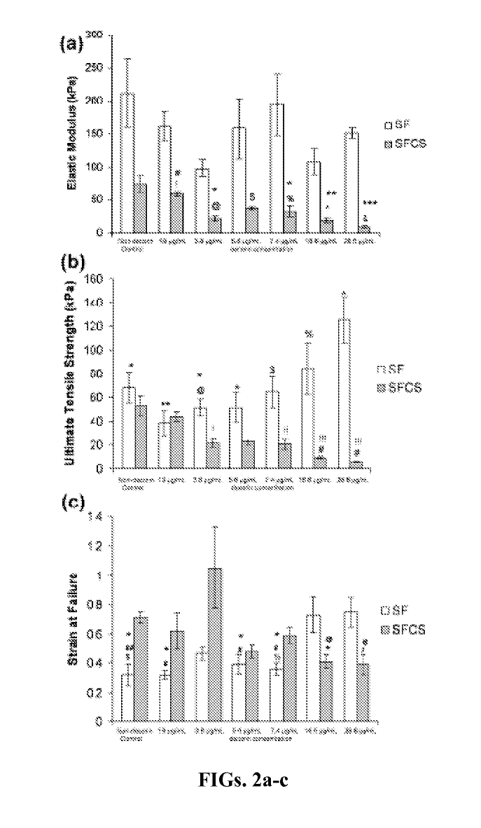

The elastic modulus, UTS, and strain at failure for each SFCS- and SF-decorin scaffold was calculated from stress strain curves. Representative stress versus strain curves for SFCS and SF scaffolds as well as SFCS- and SF-decorin scaffolds at 7.4 and 28.6 μg/mL decorin concentrations are shown in FIG. 1. FIG. 2 is a graphical representation of the average elastic modulus, UTS, and strain at failure comparing SFCS- and SF-decorin concentrations as well as the controls (SFCS and SF). The Elastic modulus of SFCS scaffolds was highest for the controls (no decorin) with significant differences against SFCS-3.8 μg/mL decorin (p<0.05), 7.4 μg/mL decorin (p<0.05), 16.6 μg/mL decorin (p<0.01) and 28.6 μg/mL decorin (p<0.001). Also, the elastic modulus of the SFCS-decorin blends decreased significantly with increasing concentrations of decorin (p<0.05, 1.9 μg/mL vs. 28.6 μg/mL decorin). At all concentrations, the elastic modulus of SFCS-decorin was significantly lower than that of SF-decorin. However, there were no significant differences between SF control and various SF-decorin blends.

Similar to the elastic modulus, the UTS of SFCS scaffolds was highest for control and decreased significantly with increasing concentrations of decorin (p<0.01, 1.9 μg/mL vs. 28.6 μg/mL decorin). However, the SF scaffolds showed the opposite trend of increasing UTS with an increase in decorin concentration. The maximum UTS values were found for SF-28.6 μg/mL decorin, which were significantly higher than SF control (p<0.05), SF-1.9 μg/mL decorin (p<0.01), SF-3.8 μg/mL decorin (p<0.05) and SF-5.6 μg/mL decorin (p<0.05). At all concentrations except 1.9 μg/mL and 5.6 μg/mL decorin, the UTS of SFCS-decorin was significantly lower than that of SF (p<0.05 at 3.8, 7.4, 16.6 μg/mL and p<0.01 at 28.6 μg/mL).

The strain at failure for SFCS-28.6 μg/mL decorin was found to be lowest and the difference was significant as compared to SFCS-3.8 μg/mL decorin (p<0.01) and SFCS control (p<0.01). Also, the strain at failure for SFCS controls was significantly higher than SF controls (p<0.01). Strain at failure of SF-decorin scaffolds was found to be maximum for 28.6 μg/mL decorin concentration, which was significantly higher than SF control (p<0.01) and SF-1.9, 5.6 and 7.4 μg/mL decorin (p<0.5).

Overall, an increase in decorin concentration caused a trend of increased mechanical properties of SF-decorin and decreased mechanical properties of SFCS-decorin. The decorin concentration of 28.6 μg/mL in SF scaffolds exhibited highest UTS as well as strain at failure for SF-decorin. Considering these results, it was important to further analyze the effect of even higher decorin concentrations (>28.6 μg/mL) on the mechanical properties of SF-decorin scaffold. The mechanical properties of SF-40.0 and 56.0 μg/mL decorin were then determined and compared with SF-28.6 μg/mL decorin scaffold (Table 1).

TABLE 1
Mechanical properties of SF-decorin scaffolds.

| | | Ultimate | |
|---|---|---|---|
| | Elastic | Tensile | |
| | Modulus | Strength | Strain at Failure |
| | (kPa) | (kPa) | (ε_{failure}) |
| SF- 28.6 μg/mL | 151.7 ± 7.7 | 125.4 ± 19.3 | 74.9 × 10⁻² ± 9.8 × 10⁻² |
| decorin | | | |
| SF- 40.0 μg/mL | 243.1 ± 20.9 | 94.3 ± 10.0 | 43.8 × 10⁻² ± 8.7 × 10⁻² |
| decorin | | | |
| SF- 56.0 μg/mL | 149.6 ± 20.9 | 77.9 ± 19.9 | 44.9 × 10⁻² ± 7.1 × 10⁻² |
| decorin | | | |

It was found that SF-40.0 and 56.0 μg/mL decorin scaffolds showed a trend of decreased UTS and strain at failure when compared to SF-28.6 μg/mL decorin; however, the difference was not significant. Hence, further increasing the decorin concentration (28.6 μg/mL) in SF scaffold does not improve the mechanical properties.

Structural Analysis of Scaffolds.

SEM imaging of 75:25 SFCS scaffolds (control) showed a very smooth surface. As the concentration of decorin in SFCS scaffolds increased, more ridge-like structures and folds were observed throughout the scaffold surface). The SF control scaffolds also showed smooth surface but with few fibril like structures. Presence of low concentrations of decorin (7.4 μg/mL) in SF scaffolds caused more fibrillar structure as compared to SF control. Higher concentration of decorin (28.6 μg/mL) in SF scaffolds showed an entangled fibrillar structure. At highest tested decorin concentration (56.0 μg/mL) SF-decorin scaffolds, an entangled fibrillar structure was still noted but with grape-like clusters.

Raman Spectroscopy Analysis.

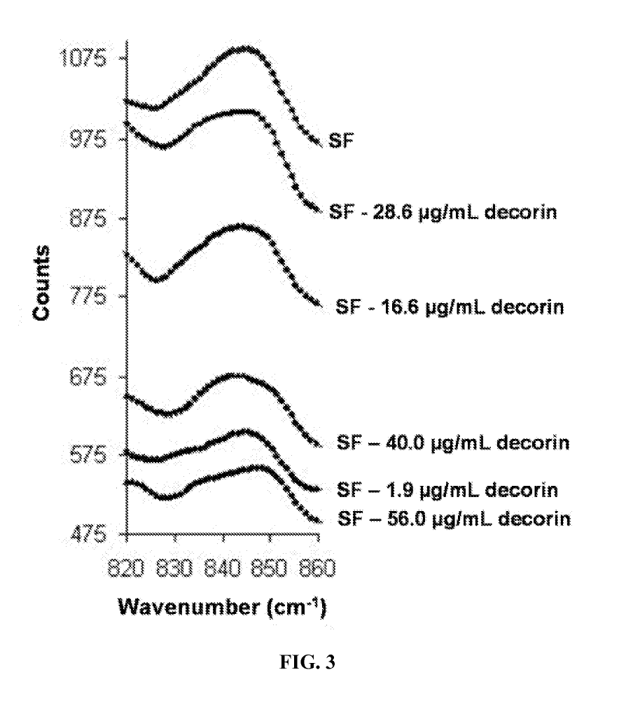

In the Raman spectroscopic analysis, the peak intensities (I) at wavenumber (cm⁻¹) 830 and 850, and their ratio as I₈₃₀/I₈₅₀ were examined. According to previous studies (Hubbell, 2003; Rosso et al., 2004; Danielson et al., 1997), increasing the I₈₅₀ represents a more random coil formation of the silk fibroin as opposed to the anti-parallel β-sheet conformation at I₈₃₀. FIG. 3 showed the Raman spectra of SF and varying concentrations of decorin in SF from wavenumbers 820-860. The wavenumbers of the 850 peaks became higher with increasing decorin concentrations in SF, indicating more random coil formation. The I₈₃₀/I₈₅₀ intensity ratios for SF and varying concentrations of decorin in SF ware calculated as seen in Table 2.

TABLE 2
I₈₃₀/I₈₅₀ intensity ratios as analyzed by Raman
spectroscopy for SF-decorin scaffolds.

| | I₈₃₀/I₈₅₀ intensity ratios |
|---|---|
| SF | 0.29 |
| SF- 1.9 μg/mL decorin | 0.27 |
| SF- 16.6 μg/mL decorin | 0.23 |
| SF- 28.6 μg/mL decorin | 0.26 |
| SF- 40.0 μg/mL decorin | 0.13 |
| SF- 56.0 μg/mL decorin | 0.11 |

This I₈₃₀/I₈₅₀ ratio for SF control was found to be 0.29, which remains almost same up to 28.6 μg/mL decorin concentration in SF. The I₈₃₀/I₈₅₀ intensity ratio decreased significantly for SF-40.0 decorin and SF-56.0 decorin scaffolds, signifying an increase in random coil structure.

All of the scaffolds and methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Gobin, A. S.; Butler, C. E.; Mathur, A. B. Tissue Engineering 2006, 12, 3383-3394.
2. Butler, C. E. Clinics in Plastic Surgery 2006, 33, 199-211.
3. Butler, C. E.; Navarro, F. A.; Orgill, D. P. Journal of Biomedical Materials Research 2001, 58, 75-80.
4. Butler, C. E.; Prieto, V. Plastic and Reconstructive Surgery 2004, 114, 464-473.
5. Butler, C. E.; Langstein, H.; S J, K. Plastic and Reconstructive Surgery 2005, 116, 1263-1275.
6. Gobin, A. S.; Froude, V. E.; Mathur, A. B. Journal of Biomedical Materials Research Part A 2005, 74A, 465-473.
7. Iozzo, R. V. Annual Review of Biochemistry 1998, 67, 609-652.
8. Reed, C. C.; Iozzo, R. V. Glycoconjugate Journal 2002, 19, 249-255.
9. Ferdous, Z.; Grande-Allen, K. J. Tissue Engineering 2007, 13, 1893-1904.
10. Vesentini, S.; Redaelli, A.; Montevecchi, F. M. Journal of Biomechanics 2005, 38, 433-443.
11. Asakura, T.; Sugino, R.; Yao, J.; Takashima, H.; Kishore, R. Biochemistry 2002, 41, 4415-4424.
12. Paola Taddei, T. A. J. Y. P. M. Biopolymers 2004, 75, 314-324.
13. Rousseau, M.-E.; Lefevre, T.; Beaulieu, L.; Asakura, T.; Pezolet, M. Biomacromolecules 2004, 5, 2247-2257.
14. Hubbell, J. A. Current Opinion in Biotechnology 2003, 14, 551-558.
15. Francesco, R.; Giordano, A.; Barbarisi, M.; Barbarisi, A. Journal of Cellular
Physiology 2004, 199, 174-180.
16. Danielson, K. G.; Baribault, H.; Holmes, D. F.; Graham, H.; Kadler, K. E.; Iozzo, R. V. The Journal of Cell Biology 1997, 136, 729-743.
17. Douglas, T.; Heinemann, S.; Bierbaum, S.; Scharnweber, D.; Worch, H. Biomacromolecules 2006, 7, 2388-2393.
18. Iwasaki, S.; Hosaka, Y.; Iwasaki, T.; Yamamoto, K.; Nagayasu, A.; Ueda, H.; Kokai, Y.; Takehana, K. Archives of Histology and Cytology 2008, 71, 37-44.
19. Weis, S. M.; Zimmerman, S. D.; Shah, M.; Covell, J. W.; Omens, J. H.; Ross, J. J.; Dalton, N.; Jones, Y.; Reed, C. C.; Iozzo, R. V.; McCulloch, A. D. Matrix Biology 2005, 24, 313-324.
20. Ferdous, Z.; Wei, V. M.; Iozzo, R.; Hook, M.; Grande-Allen, K. J. The Journal of Biological Chemistry 2007, 282, 35887-35898.
21. Iozzo, R. V. The Journal of Biological Chemistry 1999, 274, 18843-18846.
22. Iozzo, R. V.; Moscatello, D. K.; McQuillan, D. J.; Eichstetter, I. The Journal of Biological Chemistry 1999, 274, 4489-4492.
23. Macri, L.; Silverstein, D.; Clark, R. A. F. Advanced Drug Delivery Reviews 2007, 59, 1366-1381.
24. Schönherr, E.; Broszat, M.; Brandan, E.; Bruckner, P.; Kresse, H. Archives of Biochemistry and Biophysics 1998, 355, 241-248.
25. Tufvesson, E.; Westergren-Thorsson, G. FEBS Letters 2002, 530, 124-128.
26. Goldoni, S.; Seidler, D. G.; Heath, J.; Fassan, M.; Baffa, R.; Thakur, M. L.; Owens, R. T.; McQuillan, D. J.; Iozzo, R. V. The American Journal of Pathology 2008, 173, 844-855.
27. Grant, D. S.; Yenisey, C.; Rose, R. W.; Tootell, M.; Santra, M.; Iozzo, R. V. Oncogene 2002, 21, 4765-4777.
28. Matsumine, A.; Shintani, K.; Kusuzaki, K.; Matsubara, T.; Satonaka, H.; Wakabayashi, T.; Iino, T.; Uchida, A. Journal of Surgical Oncology 2007, 96, 411-418.
29. Reed, C. C.; Waterhouse, A.; Kirby, S.; Kay, P.; Owens, R. T.; McQuillan, D. J.; Iozzo, R. V. Oncogene 2004, 24, 1104-1110.
30. Rykova, V.; Grigorieva, E.; Chernenko, A.; Eshenko, T.; Dymshits, G. Bulletin of Experimental Biology and Medicine 2007, 3, 335-337.
31. Salomaki, H. H.; Sainio, A. O.; Soderstrom, M.; Pakkanen, S.; Laine, J.; Jarvelainen, H. T. Journal of Histochemistry and Cytochemistry 2008, 56, 639-646.
32. Seidler, D. G.; Goldoni, S.; Agnew, C.; Cardi, C.; Thakur, M. L.; Owens, R. T.; McQuillan, D. J.; Iozzo, R. V. The Journal of Biological Chemistry 2006, 281, 26408-26418.
33. Shintani, K.; Matsumine, A.; Kusuzaki, K.; Morikawa, J.; Matsubara, T.; Wakabayashi, T.; Araki, K.; Satonaka, H.; Wakabayashi, H.; Iino, T.; Uchida, A. Oncology Reports 2008, 6, 1533-1539.
34. Ameye, L.; Young, M. F. Glycobiology 2002, 12, 107R-116.
35. Douglas, T.; Hempel, U.; Mietrach, C.; Heinemann, S.; Scharnweber, D.; Worch, H. Biomolecular Engineering 2007, 24, 455-458.
36. Douglas, T.; Hempel, U.; Mietrach, C.; Viola, M.; Vigetti, D.; Heinemann, S.; Bierbaum, S.; Scharnweber, D.; Worch, H. Journal of Biomedical Materials Research Part A 2008, 84A, 805-816.

1. A biocompatible scaffold, comprising a silk fibroin polypeptide and a decorin proteoglycan in contact with the silk fibroin polypeptide.

2. The scaffold of claim 1, wherein the ratio of decorin proteoglycan:silk fibroin polypeptide in the scaffold ranges from about 1:100 (w/w) to about 1:1×108 (w/w).

3. The scaffold of claim 2, wherein the ratio of decorin proteoglycan:silk fibroin polypeptide in the scaffold ranges from about 1:100 (w/w) to about 1:1×106 (w/w).

4. The scaffold of claim 3, wherein the ratio of decorin proteoglycan:silk fibroin polypeptide in the scaffold ranges from about 1:100 (w/w) to about 1:1×104 (w/w).

5. The scaffold of claim 4, wherein the ratio of decorin proteoglycan:silk fibroin polypeptide in the scaffold ranges from about 1:100 (w/w) to about 1:1000 (w/w).

6. The scaffold of claim 1, further comprising a therapeutic agent.

7. The scaffold of claim 6, wherein the therapeutic agent is selected from the group consisting of an antimicrobial agent, an anti-inflammatory agent, an immunosuppressant, or a growth factor.

8. The scaffold of claim 1, wherein the silk fibroin polypeptide comprises 20 amino acids of SEQ ID NO:1.

9. The scaffold of claim 8, wherein the silk fibroin polypeptide comprises 50 amino acids of SEQ ID NO:1.

10. The scaffold of claim 9, wherein the silk fibroin polypeptide comprises 100 amino acids of SEQ ID NO:1.

11. The scaffold of claim 10, wherein the silk fibroin polypeptide comprises SEQ ID NO:1.

12. The scaffold of claim 1, wherein the scaffold has a thickness of between about 0.1 mm and about 5 mm.

13. A method of making a biocompatible scaffold, comprising:
(a) preparing a composition comprising a silk fibroin polypeptide, a decorin proteoglycan, and a solvent to create a blend;
(b) placing the blend onto a surface; and
(c) drying the blend to remove some or all of the solvent, wherein a biocompatible scaffold is formed.

14. The method of claim 13, further comprising (d) contacting the scaffold of (c) with a composition comprising an alcohol.

15. The method of claim 14, wherein the alcohol is methanol.

16. The method of claim 13, further comprising contacting the scaffold of (d) with phosphate buffered saline.

17. Use of a scaffold of claim 1 in the preparation of a medicament for treating a tissue defect in a subject.

18. The use of claim 17, wherein the tissue defect is a musculofascia defect of the abdominal wall.

19. The use of claim 17, wherein the subject is a human.

20. The use of claim 17, further comprising implanting the scaffold in a subject as part of a surgical procedure directed at repairing a musculofascia defect in a subject.

21. A kit comprising a scaffold of claim 1 in a sealed container.


SEQUENCE LISTING (11 sequences)

SEQ ID NO: 1 - protein, 262 aa, Bombyx mori
MKPIFLVLLVATSAYAAPSVTINQYSDNEIPRDIDDGKASSVISRAWDYVDDTDKSIAILNVQEILKDMASQGDYASQASAVAQTAGIIAHLSAGIPGDACAAANVINSYTDGVRSGNFAGFRQSLGPFFGHVGQNLNLINQLVINPGQLRYSVGPALGCAGGGRIYDFEAAWDAILASSDSGFLNEEYCIVKRLYNSRNSQSNNIAAYITAHLLPPVAQVFHQSAGSITDLLRGVGNGNDATGLVANAQRYIAQAASQVHV

SEQ ID NO: 2 - protein, 251 aa, Antipaluria urichi
SGSGSGAGSGSGAGSGSGAGSGSGAGSGSGAGSGSGAGSGAGSGSGAGSGSGAGSGSGAGSGSGAGSGSGSGSGSGSGSGAGSGSGSGAGSGAGSGSGAGSGSGSGSGSGAGSGSGSGAGSGSGAGSGSGSGAGSGSGSGSGSGSGSGSGSGAGSGAGSGAGSGSGAGSGSGSGSGAGSGSGSGSGSGSGSGVGSGAGSGSGSGSGSGSGSGSGSGGDNEADSGEIDCPVPADLYDSNDWDAIDAYVERYC

SEQ ID NO: 3 - protein, 443 aa, Oecophylla smaragdina
MKIPAILATSLFVWGLVGASELVGSDASATASAEASASSSAYGSKYGIGSGAVSGASASASASASASASASSAPAIEGVNVGTGVSNTASASAEALSRGLGIGQAAAEAQAAAAGQAAIAAKSCALAAKSTAQAVALVEKVARAEVDLAESARKATRLSAEAAKAAAEVEKDLVGLRGAAGKLNLAARAGSKAQERANEDSIEANELAQATAAAGAEAEAKANAAQEAGASALAIAQAALNIEQETVKLTRQAQNTRLRSENILAAASNARAIASAEAEASSDLNNRANAARSNARAAAETRAVATEAASTAEIAAYSSSEKGEITNPGPLPKIVSVTAGLTQNEIAGSGAAASASASALASASAGAGAGAGAGAGASAGAGAVAGAGAGAGAGASAGASAGANAGAGASSLLLPQSKLHPISRSSASASASAEAEANSSAYA

SEQ ID NO: 4 - protein, 400 aa, Oecophylla smaragdina
MKIPAIFVTSLLAWGLASGGVIGPDTSSSSQASASASASASASASSSASIGYNELHKSINAPALAVGVKNGGVDVAKGAAVVESAISDVSTLTDDRTLNGLAIIGNSAESLARAQASSSASAGAKANALIKQSIAAIEITEKAEYLASIVATKAAKAAEATAAATARATAVAEAAKVSSEQFAAEARAAADAEAKANAASIIANKANAVLAEAATGLSASAGKAQQSATRALQAARAAAKAQAELTQKAAQILVLIAEAKAAVSRASADQSVCTSQAQAASQIQSRASAAESAASAQSEANTIAAEAVARADAEAASQAQAWAESFKRELSSVVLEAEANASASASAGALASGSSSSGASSSADASAGASSYGSLGGYRHGGSFSEASAAASAASRAEAA

SEQ ID NO: 5 - protein, 441 aa, Myrmecia forficata
MKIPAILATSLLIWGLVGASELESEASAAASAQAEASSSGRSGKLSASQASASASASASAGSRGGSKGGWGQLRRGDVKSEAKSAAAIAVEGAKIGTGIGNTASASAEALSRGLGIGQAAAEAQAAAAGQAEVAAKSCELADKTTAKAVAMVEAAAEAEIEVANQEVAAVKLSTWAAKAARIVEEDSAAVRAAAGKLLLAARAAAAAERRANEESEAANELAQASSAAAAEAEAKANAGREAAAAALAIAEAAVAIEQEAVILARKAQDARLNAEAAAAAAMNARVIASAESEASEDLENRASVARASAAGAAEAKAIATDAGATAEIAAYSWAKKGELINPGPLPKIISVNADLSKSEVEAMKITRGQVQEVKKISTHKGGWGWGKEGRSKVSSNASARASASANAAAGSLGSKWGRQLSASSASADANAEADSQLLKVW

SEQ ID NO: 6 - protein, 357 aa, Bombus terrestris
MKIPSILAVSLLVWGLASAGKPLIANAQIGKVKTETSSSSEIETLVSGSQTLVAGSETLASESEALASKSEALTSEAEIASVTTKDELILKGEAITGKKLGTGASEVAAASGEAIATTLGAGQAAAEAQAAAAAQAKSAAAAAANAGESSNSAAALVAAAAAAQGKAAAAAAAATKASLEAADAAEEAESAVALARAASAKAEALASTAAAANTRAALQAEKSNELAQAEAAAAAEAQAKAAAAAKATQLALKVAETAVKTEADAAAAAVAAAKARAVADAAASRATAVNAIAEAEERDSAQAENTAGVAQAALAAAEAQDSCIGAAATPRHSSSYAWWKLRITSLIVILSPRNRRT

SEQ ID NO: 7 - protein, 347 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVVYLHNNNISVVGSSDFCPPGHNTKKASYSGVSLFSNPVQYWEIQPSTFRCVYVRSAIQLGNYK

SEQ ID NO: 8 - protein, 70 aa, Homo sapiens
MKATIILLLLAQVSWAGPFQQRGLFDFMLEDEASGIGPEVPDDRDFEPSLGPVCPFRCQCHLRVVQCSDL

SEQ ID NO: 9 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 10 - protein, 359 aa, Homo sapiens; identical to SEQ ID NO: 9

SEQ ID NO: 11 - protein, 359 aa, Homo sapiens; identical to SEQ ID NO: 9